(19) United States
(12) Patent Application Publication
(10) Pub. No.: US 2009/0099102 A1
(43) Pub. Date: Apr. 16, 2009

(54) Ginkgolides in the Treatment and Prevention of Ovarian Cancer

(75) Inventors: Bin Ye, Brookline, MA (US); Daniel Cramer, Chestnut Hill, MA (US)

Correspondence Address: LAW OFFICE OF MICHAEL A. SANZO, LLC, 15400 CALHOUN DR., SUITE 125, ROCKVILLE, MD, 20855, US

(73) Assignee: The Brigham and Women's Hospital, Inc., Boston, MA (US)

(21) Appl. No.: 12/084,122

(22) PCT Filed: Oct. 24, 2006
(86) PCT No.: PCT/US2006/041465

§ 371 Date: Aug. 14, 2008

Related U.S. Application Data
(60) Provisional application No. 60/730,374, filed on Oct. 27, 2005.
(60) Provisional application No. 60/735,855, filed on Nov. 14, 2005.

(51) Int. Cl. A61K 31/7028; A61K 31/343; A61P 35/00

(52) U.S. Cl. 514/25; 514/468

(57) ABSTRACT

The present invention is directed to pharmaceutical compositions and nutritional supplements that contain large amount of ginkgolides, particularly ginkgolide A or B, and for which, the other components normally found in Ginkgo biloba extracts have been removed. The compositions may be administered to women to prevent or treat ovarian cancer, particularly non-mucinous ovarian cancer.

### CROSS REFERENCE TO RELATED APPLICATIONS

The present application claims priority to and the benefit of U.S. provisional application 60/6735,855, filed on Nov. 14, 2005 and U.S. provisional application 60/730,374, filed on Oct. 27, 2005, which are hereby incorporated herein by reference in their entirety.

### STATEMENT OF GOVERNMENT FUNDING

The United States Government has a paid-up license in this invention and the right in limited circumstances to require the patent owner to license others under reasonable terms as provided for by the terms of NIH ovarian cancer case-control study (R01-CA054419-13) and Grant No. 1P50CA105009 (Ovarian SPORE) of the National Cancer Institute, awarded by the Department of Health and Human Services.

### FIELD OF THE INVENTION

The present invention is directed to compounds derived from the herb ginkgo biloba. More specifically, it is directed to pharmaceutical compositions containing ginkgolides and the use of these compositions in the treatment and prevention of ovarian cancer.

### BACKGROUND OF THE INVENTION

Plant (phyto) chemicals have received a great deal of attention in recent years for their ability to reverse, suppress or prevent carcinogenic progression to invasive cancers (Surh, Nat. Rev. Cancer 3:768-780 (2003)). Many of the anticancer effects of phytochemicals have been attributed to their anti-oxidant and anti-lipoperoxidative properties. Both of these properties are exhibited by extracts from the leaves of Ginkgo Biloba, which contain both flavonoid and terpenoid constituents (see generally published United States applications 2004/0180105 and 2001/0055629).

Although Ginkgo biloba has been widely taken as a treatment for Alzheimer's disease and peripheral arterial disease (DeFeudis, Pharmacopsychiatry, 36 Suppl 1:S2-7 (2003); Blume, et al., Vasa, 25:265-274 (1996); Schneider, Arzneimittelforschung 42:428-436 (1992)) very little attention has been paid to its use for the prevention or treatment of cancer (DeFeudis, et al., Fundam. Clin. Pharmacol. 17:405-417, 2003)). This may be due to multiple factors, including: 1) the complexity of the chemicals in the extract; 2) inconsistencies in the way in which extracts are obtained and dosages used between laboratories; 3) potential adverse interactions with other anti-cancer agents; and 4) the heterogeneity and complexity of cancers. Moreover, lack of strong epidemiological evidence may have significantly hampered enthusiasm for bench experiments and clinical trials.

Nevertheless, there are a few reports relating to cancer. Ginkgo biloba has been found to inhibit a nuclear factor called peripheral-type benzodiazepin receptor (PBR) in breast cancer cells (Papadopoulos, et al., Anticancer. Res. 20:2835-2847 (2000)) and to have a significant effect on the proliferation of human hepatocellular carcinoma (HCC) cells (Chao, et al., World J. Gastroenterol. 10:37-41, 2004)). In addition studies conducted both in vivo and in vitro have revealed that an extract of Ginkgo biloba may have anti-cancer activity related to an antioxidant inhibition of the nitric oxide (NO) pathway, anti-angiogenic and gene regulatory bioactivities (DeFeudis, et al., Fundam. Clin. Pharmacol. 17:405-417 (2003); Papadopoulos, et al., Anticancer Res, 20: 2835-2847 (2000); Chao, et al., World J. Gastroenterol. 10:37-41 (2004); Kobuchi, et al., Biochem. Pharmacol. 53:897-903 (1997)).

### SUMMARY OF THE INVENTION

The present invention is based upon both epidemiological and biological evidence indicating that Ginkgo biloba, and, in particular, the ginkgolide compounds found within this herb, reduce the risk of women developing ovarian cancer. Although protective effects can be obtained using relatively impure extracts, more highly concentrated and purified preparations are preferred, not only for greater efficacy, but also for a reduced likelihood of producing adverse side effects.

In its first aspect, the invention is directed to a pharmaceutical composition or nutritional supplement in unit dose form (preferably a tablet or capsule) comprising at least 5 mg and preferably at least 10 mg of ginkgolide. Unless otherwise indicated, the term “ginkgolide” refers to any of the ginkgolides commonly found in Ginkgo biloba and Ginkgo biloba extracts, especially ginkgolide A, B, C and J. It will also be recognized by those of skill in the art that derivatives of these compounds that are known in the art and that are equivalent in terms of structure and function may also be used in the methods discussed herein (see for examples, compounds such as BN52021, Wittwer, et al, J. Heart Lung Transplant. 20:358-63 (2001); see also Pure Appl. Chem. 71:(6):1153-1156). The terms “pharmaceutical composition” and “nutritional supplement” refer to compositions that are suitable for administration to an individual (i.e., they are nontoxic and safe) and which produce a beneficial biological effect. The difference is that pharmaceutical compositions are generally thought of as being administered to patients having a disease or disorder, whereas nutritional supplements may be given either to these individuals or to individuals that are healthy.

The term “unit dose form” refers to a single drug or supplement administration entity, e.g., a single tablet, or capsule for oral administration or vial for injection. One or more unit doses should provide sufficient ginkgolide to achieve a desired biological effect, e.g., a reduction in the risk of ovarian cancer occurring or recurring in an individual. The compositions should preferably be in a “purified” state. Unless context indicates otherwise, the term “purified” means that a ginkgolide has been separated from the other native components found in Gingko biloba leaves or the environment in which the ginkgolide was produced, found or synthesized. A “purified” preparation should have, at least 80% of these other native components removed and preferably at least 95%, 98% or 99%.

The degree of purification of ginkgolide in a pharmaceutical composition or nutritional supplement may also be expressed by reference to a reduced amount of one or more specific contaminating components. For example, a composition may contain less than 20% of ginkgoflavonglycosides and less than 5% of terpene lactones other than ginkgolide A, B, C or J. More preferred compositions have less than 10% or less than 5% of ginkgoflavonglycosides and less than 3% or 1% of terpene lactones other than ginkgolides A, B, C or J.

Although a protective effect with respect to ovarian cancer may be obtained with compositions containing lower amounts, it is preferred that compositions, other than those designed for injection, contain at least 25 mg of ginkgolide with an amount in the range of 25-100 mg being typical. In dosage forms intended for injection, 1-500 μg of purified ginkgolide should generally be present in a sterile liquid medium in which it is dissolved, suspended or emulsified. Preferably, these compositions will have 1-50 μg of ginkgolide. In these preparations, and all of the other preparations and methods discussed herein, the preferred ginkgolides are ginkgolide A and/or ginkgolide B.

In another aspect, the invention is directed to a method of treating or preventing ovarian cancer in a woman by administering an effective amount of a pharmaceutical composition or nutritional supplement in unit dose form (preferably a tablet or capsule) having at least 1 mg of ginkgolide (particularly ginkgolide A, B, C or J) and less than 20% of ginkgoflavonglycosides. The term “effective amount” or “therapeutically effective amount,” as used herein, refers to an amount of a composition sufficient to achieve a desired biological effect, in this case, a sufficient amount of ginkgolide to reduce the likelihood of a woman developing ovarian cancer relative to a woman that does not take the pharmaceutical composition or nutritional supplement. Preferred compositions for oral administration have at least 5 mg of ginkgolide and less than 20% (preferably less than 10%) of ginkgoflavonglycosides and less than 5% (preferably less than 3%) of terpene lactones other than ginkgolide A, B, C or J. For method treatments involving the injection of compositions, preparations should contain 1-500 μg of purified ginkgolide dissolved, emulsified or suspended in a liquid vehicle and this treatment should be given to a patient with non-mucinous ovarian cancer. In a preferred embodiment, the invention is directed to a method of treating or preventing ovarian cancer by administering an effective amount of any of the pharmaceutical compositions that are discussed above. As previously mentioned, the preferred ginkgolides are A and B, with the next most preferred ginkgolides being C and J.

### BRIEF DESCRIPTION OF THE DRAWINGS

FIG. 1: FIG. 1 shows the basic ring structure of the ginkgolides. For ginkgolide A: R₁=OH, R₂=H, and R₃=H. For ginkgolide B: R₁=OH, R₂=OH, and R₃=H.

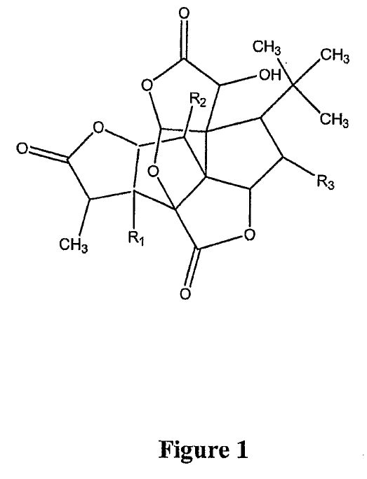

### DETAILED DESCRIPTION OF THE INVENTION

### A. Ginkgolides

The compositions and methods of the present invention may use any ginkgolide derived from ginko biloba or their equivalents, especially ginkgolides A, B, C or J, with the most preferred ginkgolides being A and B. These ginkgolides are commercially available from vendors both in the U.S (Sigma-Aldrich, Co., St. Louis, Mo.; Grace Davison Co., a subsidiary of W.R. Grace, Columbia, Md.; and Axxora, LLC, San Diego, Calif.) and elsewhere (Tauto Biochem., Shanghai, China). Alternatively, published procedures may be used for chemically synthesizing either ginkgolide A (Corey, et al., Tetrahedron Lett. 29:3205-3208 (1988)) or ginkgolide B (Corey, et al., J. Am. Chem. Soc. 110:649-651 (1988); Crimmins, et al., J. Am. Chem. Soc. 121:10249-10250 (1999)). Procedures for purifying and analyzing the ginkgolides have also been published (Pietta, et al., Chromatographia 29:51 (1990); Van Beek, et al., J. Chrom. 543:375-387 (1991)).

### B. Making of Pharmaceutical Compositions and Nutritional Supplements

Ginkgolides may be incorporated into pharmacologically active compositions or nutritional supplements made in accordance with methods that are standard in the art (see e.g., Remington's Pharmaceutical Sciences (Osol, A, ed., Mack Publishing Co., (1990)). Any of the commonly used excipients may be included in preparations. Examples of carriers include, but are not limited to: water; salt solutions; alcohols; gum arabic; vegetable oils; benzyl alcohols; polyethylene glycols; gelatin; carbohydrates such as lactose, amylose or starch; magnesium stearate; talc; silicic acid; viscous paraffin; perfume oil; fatty acid esters; hydroxymethylcellulose; polyvinyl pyrrolidone, etc. The pharmaceutical preparations can be sterilized and, if desired, mixed with auxiliary agents such as: dispersants; lubricants; preservatives; stabilizers; wetting agents; emulsifiers; salts for influencing osmotic pressure; buffers; coloring agents; flavoring agents; and/or aromatic substances.

The compositions of this invention will be dispensed in a unit dosage form generally comprising one or more active compounds in a pharmaceutically acceptable carrier. In general the dosage for a given patient should be sufficient to maintain a serum level of at least 4 ng/ml but a level of at least 8 ng/ml would be more desirable. In particular a plasma range of 8-100 ng/ml should be sufficient to reduce the risk of ovarian cancer occurring or recurring but dosages outside of this range may also be used if desired, e.g., much higher plasma levels may ultimately prove to provide better protection. When treating a patient with ovarian cancer, a physician may choose to inject preparations at a dosage that will be determined based upon clinical considerations using methods well known in the art. Typically, preparations designed for oral administration should contain 1-500 mg (preferably 10-200 or 25-100 mg) of ginkgolide. Preparations for injection should be sterile and should typically contain 1-1000 μg of ginkgolide.

Solutions, particularly solutions for injection, can be prepared using water or physiologically compatible organic solvents such as ethanol, 1,2-propylene glycol, polyglycols, dimethyl sulfoxides, fatty alcohols, triglycerides, partial esters of glycerine and the like. The preparations can be made using conventional techniques and may include sterile isotonic saline, water, 1,3-butanediol, ethanol, 1,2-propylene glycol, polyglycols mixed with water, Ringer's solution, etc. Preparations may also include preservatives.

### C. Dosage Forms and Routes of Administration

The present invention is compatible with any route of administration, including oral, peroral, internal, rectal, nasal, lingual, transdermal, vaginal, intravenous, intraarterial, intramuscular, intraperitoneal, intracutaneous and subcutaneous routes. Dosage forms that may be used include tablets, capsules, powders, aerosols, suppositories, skin patches, parenterals, sustained release preparations, and oral liquids including suspensions, solutions and emulsions. If desired, compositions may be freeze-dried and the lyophilizates reconstituted before administration, e.g., by injection. All dosage forms may be prepared using methods that are standard in the art and that are taught in reference works such as Remington's Pharmaceutical Sciences (Osol, A, ed., Mack Publishing Co. (1990)).

The ginkgolides may be used as either the sole active ingredient or in combination with other active agents such as antioxidants or flavonoids. Active ingredients may be used in conjunction with any of the vehicles and excipients commonly employed in pharmaceutical compositions, e.g., talc, gum arabic, lactose, starch, magnesium stearate, cocoa butter, aqueous or non-aqueous solvents, oils, paraffin derivatives, glycols, etc. Coloring agents, flavoring agents, aromatics, dispersants, binders, plasticizers, polymers, coatings and preservatives may also be added to preparations designed for oral administration.

Although the present invention is compatible with any route of administration and dosage form, dosage forms for oral delivery will, in most instances, be preferred, with the most preferred dosage forms being tablets or capsules. Methods for making tablets are well known in the art and procedures for making coordinated dosage forms in which agents are sequentially released have been previously described (see e.g., U.S. Pat. No. 6,479,551). In a multilayer configuration, one portion of a tablet or capsule will contain ginkgolide and other portions may contain other drugs or additional ginkgolide (e.g., for release at a different time), along with appropriate excipients, dissolution agents, lubricants, fillers, etc. Tablets may be granulated by methods such as slugging, low- or high-sheer granulation, wet granulation, or fluidized-bed granulation. Of these processes, slugging generally produces tablets of less hardness and greater friability. Low-sheer granulation, high-sheer granulation, wet granulation and fluidized bed granulation generally produce harder, less friable tablets.

If desired, enteric coating layers or a timed release formulation may be incorporated into tablets or capsules. Coating materials may include one or more of the following: methacrylic acid copolymers, shellac, hydroxypropylmethylcellulose phthalate, polyvinyl acetate phthalate, hydroxypropylmethylcellulose trimellitate, carboxymethylethylcellulose, cellulose acetate phthalate, or other suitable enteric coating polymers. The pH and time after ingestion at which coatings will dissolve can be controlled by the polymer or combination of polymers selected and/or ratio of pendant groups. For example, dissolution characteristics may be affected by the ratio of free carboxyl groups to ester groups. Coating layers may also contain pharmaceutically acceptable plasticizers such as triethyl citrate, dibutyl phthalate, tricetin, polyethylene glycols, polysorbates, etc. As mentioned previously other components, such as dispersants, colorants, anti-adhering and anti-foaming agents, may also be included.

### D. Treatment Methods

The therapeutic objective of this invention is to reduce the likelihood of a woman that is cancer free developing ovarian cancer in the future or the likelihood of an ovarian cancer patient suffering a recurrence or progression of the disease. Based upon the studies described in the examples section, these therapeutic objectives will require that enough ginkgolide be delivered to a patient to maintain a minimum serum concentration above 4 ng/ml. Higher concentrations (above 8 ng/ml, 20 ng/ml or 40 ng/ml) are preferred. When given to prevent occurrence or recurrence of ovarian cancer, these serum levels should be maintained throughout a woman's life. Thus, the compositions described herein will need to be taken at regular intervals, preferably once or twice a day. In order to determine whether an adequate level of ginkgolide is being maintained, assays of serum samples may be carried out using procedures that are known in the art.

In some instances a physician may want to administer ginkgolide compositions to ovarian cancer patients by injection. This may be done to rapidly obtain a high serum concentration and may involve either a single injection or multiple injections given regularly over a period of weeks or months. The dosage to be given will be selected by the physician based on clinical considerations using methods that are well known in the art, Guidance concerning the effect of the treatment and the effectiveness of a particular dosage may be obtained based upon a clinical evaluation whether disease progression has been slowed or stopped. If desired, patients treated with ginkgolide may concurrently receive other treatments and other therapeutic agents.

Besides being used to prevent or treat ovarian cancer, it is expected that the ginkgolide compositions described herein may also be useful in treating other cancers as well. Examples of cancers that may be treated include beast cancer, endometrial cancer, prostate cancer, lung cancer and colon cancer. The compositions may also prove useful in treating or preventing neurological conditions such as Alzheimer's disease and cardiovascular diseases.

### EXAMPLES

The present example examines the protective effects of various herbal supplements using epidemiological data. In addition, specific components of Ginkgo biloba that may affect ovarian cancer rate are examined in in vitro studies.

### I. Materials and Methods

Epidemiological Study Method

1267 cases of ovarian cancer were identified from tumor boards and statewide registries. Of these, 119 cases were excluded due to death, 110 were excluded because they had moved from the study area, 1 case was eliminate due to not having a telephone, 23 cases were eliminated because they did not speak English, and 46 cases were eliminated because they were found to have a non-ovarian primary cancer upon review. Of the remaining 968, physicians denied permission to contact 106 and 171 declined to participate leaving 691 cases interviewed. Of these, 668 had an epithelial ovarian cancer (including borderline malignancies) and are included in this report.

Controls were identified through town books in MA and Drivers' License lists in NH and sampled to match the age and residence of previously accumulated cases. Invitations to participate were sent to 1843 potential controls. Of these 318 had moved and could not be located or had died, 197 (in MA) could not be re-contacted because subjects returned an “opt out” postcard required by the hospital IRB, and 47 no longer had a working telephone. Of the remaining 1281 who were contacted, 152 were ineligible because they had no ovaries or were not the correct age, 59 were incapacitated or did not speak English, and 349 declined leaving 721 who were interviewed and included in this report.

After written informed consent, an in-person interview dealing with demographic, medical and family history was conducted. We asked about herbal remedies used continuously for six or more months. Subjects were asked about exposures prior to a reference date defined 1 year prior to their date of diagnosis for ovarian cancer cases and 1 year prior to the date of interview for controls. Subjects also completed a self-administered dietary questionnaire. Heparinized blood specimens were collected from subjects agreeing to provide one, separated into red cell, buffy coat, and plasma components, and stored at −80 centigrade.

Chemical Reagents

Dimethyl sulphoxide (DMSO), quercetin, kaempferol, ginkgolide A, and B (>90% HPLC grade) were purchased from Sigma. Standard Ginkgo biloba extract powder, with active ingredients of 24% Ginkgoflavon-glycosides, and 6% terpene lactones, was purchased from Spectrum Chemical MFG Corp. (New Brunswick, N.J.). Cell culture medium of MCDB-105 and medium 199 were purchased from Sigma-Aldrich (St. Louis, Mo.) and F12 from Invitrogen (Carlsbad, Calif.).

Cell Culture

Immortalized normal human ovary epithelial cell (HOSEE6E7) and a serous type ovarian cancer cell lines (OVCA433) previously established in our laboratory (Tsao, et al., Exp. Cell Res. 218:499-507 (1995); Kim, et al., Jama 287:1671-1679 (2002)) were used in this study. Two mucinous ovarian cancer cell lines (RMUG-S and RMUG-L) were purchased from The Japanese Collection of Research Bioresource (Tokyo, Japan). RMUG-L cells were cultured in MCDB105/M199 medium as above, and RMUG-S cells were cultured in F-12 medium with 10% Fetal Bovine Serum and 1% antibiotic as above. Cells were maintained at 37° C. under 5% CO2 and 95% air in a high humidity chamber. Monolayer cells at 60-80% confluence were enzymatically removed using trypsin/EDTA and plated in 96 well flat-bottomed plates at concentration of 5×10³ per well for HOSE-E6E7 cells, 1×10³ per well for OVCA429 and RMUG-L, respectively. Treatments were added 24 hours after plating. Ten mM stock solutions of Ginkgo biloba, quercetin, kaempferol, ginkgolide A, and ginkgolide B, were prepared in DMSO and cells were treated with 10, 50 and 100 μM concentrations of each component. Concentration of standard Ginkgo biloba treatment was calibrated according to the fact that, the standard Ginkgo biloba mixture contains about 4-6% of total active terpene lactons, and the estimated average of the molecular weight of the mixture is about 300. An equal volume of DMSO (less than <1% concentration) was used as control treatment.

Cell Proliferation, Cell Cycle and Apoptosis Analysis

Methods for the cell proliferation and viability assays were adopted from MTT (Mosmann, J. Immunol. Methods 65:55-63 (1983), XTT (Scudiero et al., Cancer Res. 48:4827-4833, (1988)) and MTS (Cory, et al., Cancer Commun. 3: 207-212 (1991)), which developed as WST-1 (Roche Applied Science, Germany). Briefly, the tetrazolium salts are cleaved to formazan by cellular enzymes. An expansion in the number of viable cells results in an increase in the overall activity of mitochondrial dehydrogenases in the sample. This augmentation in enzyme activity leads to an increase in the amount of formazan dye formed, which directly correlates with the number of metabolically active cells in the culture. After 72 hours of treatment, 15 μl of the MTT dye solution was added to each well and the plates were incubated at 37° C. for 4 hours in a humidified chamber. After incubation, 100 μl of the Solubilization/Stop solution was added to each well. One hour after the addition of the solubilization solution, the contents of the wells were mixed and read by the 96-well plate scanning spectrophotometer (μQuant) and quantitative software (KC-junior, Bio-Tek Instruments, Inc.) at an absorbance of 630 nm for quantitative analysis. Data was collected from at least 3 separate experiments, and at least 8 repeats were performed for each individual treatment. The cell proliferation rate was expressed as a percentage of the control, which had been treated with an equivalent volume of DMSO (as 100%).

For the cell cycle analysis, ovarian cells were plated in 75 cm² flasks and treated with 100 μM of ginkgolide A and B; the control was treated with an equal amount of DMSO. After 24 and 48 hours, cells were harvested by trypsin digestion and followed by PBS washing and spun for 5 min at room temperature at 12,000 rpm. Cells were fixed with 70% ethanol (in PBS buffer) by suspending the cell pellet and incubated at room temperature for 5 minutes or at −20° C. for two days. The fixed cells were washed once with PBS and then treated with RNase A (50 ug/ml) at 37° C. for 30-60 min. After a single washing with PBS to remove RNase A, propidium iodide (25 μg/ml in PBS buffer) was added. Cells were subjected to analysis using a FACS Calibur Flow Cytometer (Becton Dickson, San Jose, Calif.). Each experiment was repeated at least three times and the cell cycle profiles and data were analyzed by ModFit LT software (Verity Software House, Inc., Topsham, Me.).

LC-MS/MS Analysis and Quantification of Ginkgolides

To prepare the sample for the ginkgolide analysis, a 50 μL aliquot of human serum was diluted with 50 μL of 45% aqueous acetonitrile and then precipitated with 100 μL of 90% acetonitrile. Standard solutions containing 0.001, 0.005, 0.01, 0.1, 1 and 10 μg/mL of both ginkgolide A and ginkgolide B, respectively, were prepared in the presence of equal volume of human serum and aqueous acetonitrile as described above. All treated samples were centrifuged at 13,000 rpm for 3 minutes. The supernatants were transferred to a 96-well plate for the LC/MS/MS analysis. The LC/MS/MS system consisted of a CTC Pal auto injector (Leap Technologies, Carrboro, N.C.), a Rheos CPS-LC binary pump (Flux instruments, Basel, Switzerland), and API 4000 triple quadrupole mass spectrometer (Applied Biosystems, Foster City, Calif.). HPLC separation was performed using a Luna C18-Cartridge column (20×2.0 mm, 5 μm, Phenomenex, Torrance, Calif.). A combination of an isocratic and a linear gradient from 65% mobile phase A (100% H₂O, 0.1% formic acid, 0.01% trifluoroacetic acid (TFA) to 80% mobile phase B (0.1% formic acid, 0.01% TFA in ACN/H₂O (V/V; 90/10) over 1.5 minutes at a flow rate of 200 μL/min was used to elute ginkgolides in the serum extracts. Total run time including column re-equilibration, was 3 minutes. Specific parent/daughter ion pairs (ginkgolide A: 409.3/345.2; and ginkgolide B: 425.2/361.3) were monitored under a multiple-reaction monitoring (MRM) mode using an electrospray positive (ES+) ionization source on the API 4000.

Statistical Analysis

Logistic regression analysis was used to calculate the exposure odds ratio to estimate the relative risk (RR) for ovarian cancer associated with the use of any or a particular type of herbal remedy. Adjustment variables included age, study center, oral contraceptive (OC) use, parity, and Jewish ethnic background. Additionally, we adjusted individually for categories of consumption of vitamin A, carotene, tomatoes and tomato sauce and juice, and raw carrots based upon previous work showing the importance of these foods and vitamins on ovarian cancer risk in our data (Cramer, et al., Int. J. Cancer 94:128-134 (2001)). For the data from the cell proliferation assay, linear regression models were applied to analyze mean OD values from the MTT assays across the different concentration treatments, adjusted for individual experiments. We used a partial F test to determine whether mean OD levels varied across the four concentrations (control, 10, 50, 100 μM). If the means were found to be significantly different, we tested whether or not the treatment OD means were significantly different from the control OD mean. All analyses were conducted with SAS statistical software (SAS Institute, Inc., Cary, N.C.).

### II. Results

Epidemiological Study

Overall 80 (11.1%) of the 721 controls and 67 (10%) of the cases reported regular use of any type of herbal or nutritional supplement (p=0.71) (Table 1). While over 30 separate supplements were reported, only the five listed in table 1 were used by more than 1% of subjects. Among these five, only one type of herbal supplement was found to be significantly associated with risk for ovarian cancer, and this was Ginkgo. Thirty (4.2%) of controls versus 11 (1.6%) of cases had regularly used Ginkgo for an adjusted RR (and 95% confidence interval of 0.41 (0.20-0.84) (p=0.01). Use of Ginkgo as the only herbal remedy was also associated with a decreased risk for ovarian cancer, adjusted RR=0.36 (0.14-0.91) p=0.03. Additional adjustment of the Ginkgo association by consumption of vitamin A, carotene, tomatoes and tomato sauce and juice, and raw carrots did not alter the association. Stratifying cases by whether they had a mucinous versus non-mucinous type of tumor, we found that the inverse association of Ginkgo biloba use and ovarian cancer was confined to women with non-mucinous types of ovarian cancer RR=0.33 (0.15-0.74) (p=0.007) (Table 2). Because of the small number of exposed subjects, examination of the association by recency and duration of use was very limited (Table 3). The majority of both cases and controls who reported Ginkgo use were using it at their reference date and had used it less than two years; and these were the categories for which the association was significant. No information on the exact type of Ginkgo preparation and number of tablets taken daily was available.

In Vitro Cellular Study

A crude extract of Ginkgo biloba as well as its pure chemical components including quercetin, kaempferol, ginkgolide A and ginkgolide B, were used for treatment at different concentrations and time points in serous cancer cells (OVCA429), a mucinous type cancer cell line (RMUG-L), and HOSE-E6E7 cells. The standard Ginkgo biloba extract at concentrations equivalent to 10, 50 and 100 μM of ginkgolides had significant (p<0.01) inhibitory effects on OVCA429 cells, but much less effect was observed in HOSE and RMUG-L cells. In general, there was little effect observed with quercetin and kaempferol (except for the HOSE-E6E7 cells with 100 μM kaempferol, OVCA429 with 50 and 100 μM quercetin). Ginkgolide A at 50 and 100 μM and ginkgolide B at 10, 50 and 100 μM showed a significant (p<0.0001) effect on OVCA 429 cancer cell proliferation, which was reduced to about 60% compared to the control treatments. But little effect was observed in HOSE-E6E7 cells and mucinous type cancer cells RMUG-L. Interestingly, the effect of ginkgolide B on OVCA429 proliferation was comparable in its pattern to the effect of standard Ginkgo biloba extract. A similar anti-proliferative effect was also observed in other non-mucinous ovarian cancer cell lines such as OVCA420 and OVCA433, with about 30-40% cell proliferation inhibition by ginkgolide A and B treatment.

Cell Cycle Analysis

The effect of ginkgolide on cell cycle and DNA distribution was analyzed in HOSE-E6E7 cells, serous type OVCA429 and mucinous type RMUG-L cells. After 24 h treatment, neither ginkgolide A nor ginkgolide B (100 μM) showed a significant effect on DNA content of G0/G1, S and G2/M phases in HOSE-E6E7 cells. After 24 and 48 h treatment with ginkgolide B (100 μM) on OVCA429 cells, G0/G1 phase DNA was significantly (p<0.01) increased from 47.9% to 51.5% and 65.1%, respectively. S-phase DNA contents were decreased to 35.8% and 24% after ginkgolide B treatment compared to the control (43%) (p<0.01). However, there was a reverse effect observed in mucinous cells (RMUG-L). After 48 h treatment, the G0/G1 phase DNA was decreased from 57% to 44%, and S-phase DNA was increased from 28.7% to 40.9%. There were no observed changes in G0/G1, S-phase and M-phase DNA distribution in mucinous RMUG-S cells after treatment. Similar results were observed in OVCA429, after 48 h treatment with ginkgolide A. Cell G0/G1 DNA was increased from 50.7% to 86.1% and S-phase DNA contents was decreased from 36.5% to 10.3%.

LC-MS/MS Quantification of Ginkgolides

Because we identified that ginkgolides A and B were the most active components of Ginkgo biloba extract, we sought at least descriptive evidence that subjects who used Ginkgo biloba had detectable ginkgolides in their plasma. Using LC-MS/MS as described in Methods, we constructed standard curves of ginkgolides at 0.001 to 1 μg per ml concentrations and these were used for calibration. Up to 8.5 ng/ml of ginkgolide A and 1.0 ng/ml ginkgolide B were detected in the plasma sample of a woman, who had routinely taken Ginkgo biloba for 136 months. There were no signals detected in the plasma of a woman with no reported use of Ginkgo biloba.

### III. Discussion

We have presented epidemiological data supporting an association between regular (at least 6 months continuous) use of Ginkgo biloba and a decreased risk for ovarian cancer, and biological data supporting anti-proliferative effects of key Ginkgo components, which might underlie a chemopreventive effect. The epidemiologic association between Ginkgo use and ovarian cancer appeared to be confined to women with non-mucinous ovarian tumors and indicated that regular use was associated with a RR (and 95% CI) of 0.33 (0.15-0.74) indicating about a 67% decrease in risk for non-mucinous tumors associated with use of Ginkgo.

We asked women with ovarian cancer to focus on the use of herbal supplements at least one year prior to their cancer diagnosis, so that the addition or discontinuation of herbal supplements because of cancer symptoms or treatments was not a factor in the association. In addition, we adjusted for key potential confounders including the well-known protective factors of parity and oral contraceptive use as well as age, study center and Jewish ethnic background—a strong correlate of BRCA1 or BRAC2 cancers. Neither do we believe that the finding represents overselection of healthy controls in our study more likely to use herbal supplements since the association was specific for Ginkgo and not other commonly used remedies. In addition our finding that between 4-5% of women were using Ginkgo matches use reported by national surveys in the US (Kelly, et al., Arch. Intern. Med. 165:281-286 (2005)). The ability to examine for a dose response was limited by the small number of exposed subjects and by the fact that we did not ask about number of tablets taken daily.

Although the potential effects of Ginkgo in the prevention of circulatory disturbances have received considerable attention, its role as a cancer chemopreventive or supplement to conventional therapy is receiving more attention (DeFeudis, et al., Fundam Clin Pharmacol 17:405-417 (2003)). Ginkgo biloba and its components such as quercetin and ginkgolide may affect a number of cancers through many different pathways as illustrated by the following studies: 1) increased antioxidant activity observed against bladder and breast cancer (Papadopoulos, et al., Anticancer Res. 20:2835-2847 (2000); Kobuchi, et al., Biochem. Pharmacol. 53:897-903 (1997); Gohil, et al., Free Radic. Res. 33:831-849 (2000)); 2) inhibition of cell proliferation and induced cytotoxity in human liver cancer cells (Chao, et al., World J. Gastroenterol. 10:37-41 (2004); Cheung, et al., Biochem. Pharmacol. 61:503-510 (2001)); 3) blockage of the angiogenic response in lung cancer and decrease metastases (Juarez, et al., Eur. J. Opthalmol. 10:51-59 (2000); Monte, et al., Free Radic. Biol. Med. 17:259-266 (1994)) 4) induction of gene expression patterns associated with cell proliferation and cell cycle in breast cancer cells (Papadopoulos, et al., Anticancer Res. 20:2835-2847 (2000); Gohil, et al., Free Radic. Res 33:831-849 (2000)); 5) induced detoxification enzymes such as cytochrome P450 (CYP), glutathione S-transferase and quinone reductase to prevent colon cancer (Suzuki, et al., Cancer Lett. 210:159-69 (2004)); and 6) induction of anti-clastogenic effects in leukemia (Emerit, et al., Radiat. Res. 144:198-205 (1995)).

In our review, we were unable to identify any direct biological data, which would specifically link Ginkgo to ovarian cancer prevention. Thus we performed our own set of experiments that focused on both standardized crude Ginkgo extract as well as its purified individual components. We found that crude Ginkgo extract, its pure diterpene components of ginkgolide A and B, and its pure flavonoid quercetin have a significant inhibitory pattern in ovarian cancer cells. The fact that the antiproliferative effect of Ginkgo extract at about 2 mM (equivalent to 100 μM ginkgolide) was somewhat greater compared to the individual ginkgolides could suggest a synergistic effect of quercetin, kaempferol, ginkgolide A and B.

In addition, the anti-proliferative effect of ginkgolide A and B appeared to be, at least partially due to cell cycle blockage at G0/G1 to S phase checking point, evident in serous type cancer cells, but not on mucinous type cancer cells i.e. RMUG-L cells. It is of some interest that Wei et al. reported that ginkgolide B can inhibit smooth muscle cell proliferation in a concentration dependent manner with inhibition related to a G1 to S phase blockage in cell cycle Wei, Yao Xue Xue Bao 37:90-93 (2002)). It is not clear why ginkgolide A and B have no effect on the RMUG-L line of mucinous ovarian cancer cells (or even a reverse effect from the cell cycle). However, this observation is consistent with both data suggesting mucinous ovarian tumors do appear to differ from non-mucinous types in several ways (Pisano, et al., Anticancer Res 25:3501-3505 (2005)); Pectasides, et al., Gynecol Oncol 97:436-441 (2005); Wang, et al., Cancer Genet Cytogenet 161:170-173 (2005); Wamunyokoli, et al., Clin Cancer Res 12:690-700 (2006)).

Although independent inhibitory effects of quercetin, which commonly exist in many different diets and supplements such as St. John wort, have been reported in different types of tumor, we found that among the list of herbal supplements only Ginkgo was associated with a decreased ovarian cancer risk in our epidemiological study. Ginkgolides are unique compounds and can only be found in the Ginkgo biloba extract, therefore we believe that the inhibitory effect of Ginkgo may primarily result from its diterpene components ginkgolide A and B. In fact, many types of natural diterpenes such as paclitaxel (Gregory, et al., Clin. Pharm. 12:401-415 (1993)) and triterpenes such as 2-cyano-3,12-dioxooleana-1,9,-dien-28-oic acid (CDDO) (Ravelo, et al., Curr. Top. Med. Chem. 4:241-265 (2004); Molnar, et al., Curr. Pharm. Des. 12:287-311 (2006); Stadheim, et al., J. Biol. Chem. 277:16448-16455 (2002)) have anticancer activities.

Because our in vitro experimental data indicates the anti-proliferative effects may reside primarily with the ginkgolide components of Ginkgo extract, we thought it was important to demonstrate that this component is actually present in women who used Ginkgo. Using an LC-MS/MS based quantitative assay, up to 8.5 ng/ml of ginkgolide A and 1.0 ng/ml ginkgolide B were detected in the plasma sample of a woman, who had routinely taken Ginkgo biloba for 136 months while no signals were detected in the control plasma of a non-user. These concentrations are consistent with averages of 15 ng/ml measured for ginkgolide A and 4 ng/ml for ginkgolide B reported in 10 young healthy volunteers after a single oral dose of 80 mg of Ginkgo biloba (EGb 761 formula) (Fourtillan, et al., Therapie 50:137-144 (1995)).

TABLE 1
Distributions and Comparison of Herbal/Nutritional Supplements
in Ovarian Cancer Case-Control Population

| | | | | |
|---|---|---|---|---|
| Herbal/Nutritional | | | | |
| Supplement at Least Weekly | Cases | Controls | Adjusted* | |
| for 6 Months or Longer | N (%) | N (%) | RR (95% CI) | p-value |

Any use

| | | | | | | |
|---|---|---|---|---|---|---|
| No | 601 | (90.0) | 641 | (88.9) | 1.00 | |
| Yes | 67 | (10.0) | 80 | (11.1) | 0.94 (0.66, 1.33) | 0.71 |

Ginkgo

| | | | | | | |
|---|---|---|---|---|---|---|
| No | 657 | (98.4) | 691 | (95.8) | 1.00 | |
| Yes | 11 | (1.6) | 30 | (4.2) | 0.41 (0.20, 0.84) | 0.01 |

Echinacea

| | | | | | | |
|---|---|---|---|---|---|---|
| No | 657 | (98.4) | 709 | (98.3) | 1.00 | |
| Yes | 11 | (1.6) | 12 | (1.7) | 0.94 (0.40, 2.19) | 0.88 |

Ginseng

| | | | | | | |
|---|---|---|---|---|---|---|
| No | 663 | (99.2) | 713 | (98.9) | 1.00 | |
| Yes | 5 | (0.8) | 8 | (1.1) | 0.77 (0.25, 2.43) | 0.66 |

St. Johns Wort

| | | | | | | |
|---|---|---|---|---|---|---|
| No | 653 | (97.8) | 708 | (98.2) | 1.00 | |
| Yes | 15 | (2.2) | 13 | (1.8) | 1.26 (0.59, 2.70) | 0.55 |

Glucosamine/Chondroitin

| | | | | | | |
|---|---|---|---|---|---|---|
| No | 661 | (99.0) | 712 | (98.8) | 1.00 | |
| Yes | 7 | (1.0) | 9 | (1.2) | 0.90 (0.32, 2.52) | 0.84 |

Mutually exclusive categories

| | | | | | | |
|---|---|---|---|---|---|---|
| No Use | 601 | (90.0) | 641 | (88.9) | 1.00 | |
| Ginkgo alone | 6 | (0.9) | 19 | (2.6) | 0.36 (0.14, 0.91) | 0.03 |
| Echinacea alone | 10 | (1.5) | 10 | (1.4) | 0.95 (0.38, 2.36) | 0.92 |
| Ginseng alone | 1 | (0.2) | 4 | (0.6) | 0.35 (0.04, 3.14) | 0.34 |
| St. Johns Wort alone | 13 | (2.0) | 6 | (0.8) | 2.17 (0.81, 5.80) | 0.12 |
| Glucosamine alone | 7 | (1.0) | 7 | (1.0) | 1.24 (0.42, 3.69) | 0.70 |
| Other alone | 25 | (3.7) | 22 | (3.0) | 1.31 (0.72, 2.39) | 0.37 |
| Combination | 5 | (0.8) | 12 | (1.7) | 0.49 (0.17, 1.44) | 0.19 |

*Adjusted for age, study center, OC use, parity, and Jewish ethnic background.

TABLE 2
Ginkgo biloba Supplement Decrease Risk
Factor for Non-Mucinous Ovarian Cancers

| Disease | Any Ginkgo | | No Ginkgo | | Adjusted* | p- |
|---|---|---|---|---|---|---|
| Status | Use N (%) | | Use N (%) | | RR (95% CI) | value |
| Controls | 30 | (4.2) | 691 | (95.8) | 1.00 | |
| Cases | 11 | (1.6) | 657 | (98.4) | 0.41 (0.20, 0.84) | 0.01 |
| Mucinous | 3 | (4.1) | 70 | (95.9) | 1.17 (0.34, 4.05) | 0.81 |
| Non-mucinous | 8 | (1.3) | 587 | (98.7) | 0.33 (0.15, 0.74) | 0.007 |

*Adjusted for age, study center, OC use, parity, and Jewish ethnic background.

TABLE 3
Recency and duration of Ginkgo use among cases
with non-mucinous ovarian cancer and controls

| | | | | |
|---|---|---|---|---|
| Ginkgo use at Least Weekly | Non-mucinous | Controls | Adjusted* | |
| for 6 Months or Longer | Cases N (%) | N (%) | RR (95% CI) | p-value |

Ginkgo use at reference age

| | | | | | | |
|---|---|---|---|---|---|---|
| Never used | 587 | (98.7) | 691 | (95.8) | 1.00 | |
| No longer using | 2 | (0.3) | 9 | (1.2) | 0.25 (0.05, 1.21) | 0.09 |
| Currently using | 6 | (1.0) | 21 | (2.9) | 0.37 (0.15, 0.93) | 0.03 |

Duration of use

| | | | | | | |
|---|---|---|---|---|---|---|
| No use | 587 | (98.7) | 691 | (95.8) | 1.00 | |
| ≦2 years | 6 | (1.0) | 23 | (3.2) | 0.34 (0.14, 0.85) | 0.02 |
| >2 years | 2 | (0.3) | 7 | (1.0) | 0.31 (0.06, 1.54) | 0.15 |

*Adjusted for age, study center, OC use, parity, and Jewish ethnic background.

All references cited herein are fully incorporated by reference. Having now fully described the invention, it will be understood by those of skill in the art that the invention may be practiced within a wide and equivalent range of conditions, parameters and the like, without affecting the spirit or scope of the invention or any embodiment thereof.

1. A pharmaceutical composition or nutritional supplement in unit dose form comprising at least 10 mg of a ginkgolide.

2. The pharmaceutical composition or nutritional supplement of claim 1, wherein said pharmaceutical composition or nutritional supplement comprises less than 20% of ginkgoflavonglycosides and less than 5% of terpene lactones other than ginkgolide A, B, C or J.

3. The pharmaceutical composition or nutritional supplement of claim 1, wherein said pharmaceutical composition or nutritional supplement comprises less than 10% of ginkgoflavonglycosides and less than 3% of terpene lactones other than ginkgolide A, B, C or J.

4. The pharmaceutical composition or nutritional supplement of claim 1, wherein said pharmaceutical composition or nutritional supplement comprises less than 5% of ginkgoflavonglycosides and less than 1% terpene lactones other than ginkgolide A, B, C or J.

5. The pharmaceutical composition or nutritional supplement of claim 1, wherein said pharmaceutical composition or nutritional supplement comprises at least 25 mg of said ginkgolide.

6. The pharmaceutical composition or nutritional supplement of claim 1, wherein said pharmaceutical composition or nutritional supplement is in the form of a tablet or capsule comprising at least 10 mg of said ginkgolide and less than 20% of ginkgoflavonglycosides.

7. The pharmaceutical composition or nutritional supplement of claim 6, wherein said pharmaceutical composition or nutritional supplement comprises 25-100 mg of said ginkgolide and less than 10% of ginkgoflavonglycosides.

8. A pharmaceutical composition comprising 0.1-5000 μg of purified ginkgolide dissolved, emulsified or suspended in a sterile, pharmaceutically acceptable liquid vehicle suitable for administration to a patient by injection.

9. The pharmaceutical composition of claim 8, wherein said pharmaceutical composition comprises 1-500 μg said ginkgolide.

10. The pharmaceutical composition of claim 9, wherein said pharmaceutical composition comprises 1-50 μg of said ginkgolide.

11. The pharmaceutical composition or nutritional supplement of claim 1, wherein said ginkgolide is either ginkgolide A or ginkgolide B.

12. A method of treating or preventing ovarian cancer in a woman, comprising administering to said woman an effective amount of a pharmaceutical composition or nutritional supplement in unit dose form wherein said pharmaceutical composition or nutritional supplement comprises at least 1 mg of a ginkgolide and less than 20% of ginkgoflavonglycosides.

13. The method of claim 12, wherein said pharmaceutical composition or nutritional supplement comprises at least 5 mg of said ginkgolide, less than 20% of ginkgoflavonglycosides and less than of 5% terpene lactones other than ginkgolide A, B, C or J.

14. The method of claim 13, wherein said pharmaceutical composition or nutritional supplement comprises less than 10% of said ginkgoflavonglycosides and less than 3% of terpene lactones other than ginkgolide A, B, C or J.

15. The method of claim 12, wherein said pharmaceutical composition or nutritional supplement is administered as a treatment for non-mucinous ovarian cancer.

16. A method of treating or preventing ovarian cancer in a woman, comprising administering to said woman an effective amount of a pharmaceutical composition in unit dose form said pharmaceutical composition comprising 1-500 μg of purified ginkgolide dissolved, emulsified or suspended in a liquid vehicle and administered by injection as a treatment for non-mucinous ovarian cancer.

17. The method of claim 16, wherein said ginkgolide is either ginkgolide A or B.

18. A method of treating or preventing ovarian cancer in a woman, comprising administering to said woman an effective amount of the pharmaceutical composition of claim 1.

19. The method of claim 18, wherein said ginkgolide is either ginkgolide A or B.

20. The method of claim 18, wherein said pharmaceutical composition is administered as a treatment for non-mucinous ovarian cancer.
